(19) United States
(12) Patent Application Publication
(10) Pub. No.: US 2017/0000996 A1
(43) Pub. Date: Jan. 5, 2017

(54) Brain Portal

(71) Applicant: alan edward odonnell, asheville, NC (US)

(72) Inventor: alan edward odonnell, asheville, NC (US)

(21) Appl. No.: 14/341,271

(22) Filed: Jul. 25, 2014

(51) Int. Cl. A61M 39/02; A61N 1/05

(52) U.S. Cl. CPC A61M 39/0247; A61N 1/0529; A61M 2039/025; A61M 2039/0267; A61M 2039/0276; A61M 2207/00; A61M 2210/0687; A61M 2210/0693

(57) ABSTRACT

The Brain Portal is a device that allows for easy access to the brain for treatments that require multiple visits inside the brain case to alleviate the need for multiple craniotomies. Such treatments could involve multiple injections of medicines that have to be given over a period of days or weeks. In a different configuration, it also allows for electrical stimulation of the brain thru electrodes without the need for batteries, and allows the flow of electricity from outside the brain to the affected areas using induction coils. The Brain Portal can also be used as an easy means to collect samples of cerebral spinal fluid without the need of a spinal tap. Furthermore, having an easy access allows the physician the option of injecting drugs directly into the brain, bypassing the blood-brain barrier, thereby lowering the dosage needed to treat disease.

### BACKGROUND

For thousands of years, mankind has had to deal with head trauma. The consequence of some of these injuries resulted in swelling of the brain or its support structures, which lead to a very poor prognosis. The symptoms were clear to the Stone Age shamans as they are to the present day neurosurgeon. When there is swelling in the brain, there is no natural way for the brain to relieve the pressure, which leads ultimately to death.

It was not surprising at all that stone-age doctors attempted radical methods to try to help their patients. Cutting a hole in the brain case to relieve the swelling was a last-ditch effort to save the patient. Surprisingly, in some cases it worked. Evidence from skulls with primitive craniotomies showed healing around the edges of the hole, which indicates the patient survived the operation.

Gaining access to the brain cutting holes in the skull is still necessary for a variety of reasons. Almost all of these operations are performed by specialists (neurosurgeons) in operating rooms. The procedures have obviously improved, but the basic steps of pealing back the scalp, cutting or drilling a hole, and proceeding with whatever procedure is called for remains pretty much the same as it did in the Stone Age.

With the advent of new drug therapies to treat neuro diseases comes the problem of needing to access the brain on an industrial scale. Alzheimer's disease, Parkinson's disease, ALS, and other diseases will inflict death on over 36 million victims in the next few years, and those numbers cover just those in the United States. New drugs are on the horizon that will alleviate the symptoms of these killer diseases.

The problem with drug therapies is the blood-brain barrier which helps protect the brain from bacteria. If the drug is too big, it won't flow into the brain; no matter how much is placed in the bloodstream (toxicity levels are reached in the rest of the body before therapeutic levels are reached in the brain). Doctors need a way to inject the life-saving drugs directly into the brain.

Traditional craniotomies are not the answer. Neurosurgeons are very highly trained, and there is nowhere near the number needed to save the lives of 36 million people. Implants won't solve the problem either, as they must be carefully placed by neurosurgeons. Factoring in the other parameters of the lack of operating rooms and costs means the majority of the 36 million people will die if a simpler, cheaper and quicker means of delivery is not employed.

What is needed is a device that is cheap, safe, and effective. It must be able to be employed by a family doctor (with a small amount of additional training and tools) in a doctor's office in less than 30 minutes. The device has to allow the injection of multiple treatments, and also prevent any infections from developing from its emplacement.

### USES OF THE BRAIN PORTAL

The Brain Portal is small, inexpensive to make, can be quickly manufactured (millions can be made in a matter of months), and is designed to be easy enough to emplace that any doctor can do it in his office in about 30 minutes. It is strong enough to withstand dozens of injections without failing. It will bring hope to the millions who will surely die without treatments.

What about other diseases that require medications to be delivered directly to an affected part of the brain? Neurosurgeons will appreciate the attachment that comes with the Brain Portal. The attachment fits over a standard probe, which is used to locate the precise area that needs treatment. When the probe reaches the desired area, the probe is retracted, leaving the attachment in place which allows medication to flow from the Brain Portal down through the attachment into the diseased area.

Another embodiment of the Brain Portal will allow the transmission of electrical impulses to the brain through the use of a small induction coil. Instead of the injection membrane, a solid plug fitted with electrodes and an induction coil is present. After emplacement under the scalp and the electrodes placed in the desired location, the device can be powered by inducing electrical currents through the scalp to the device.

### BRIEF DESCRIPTION OF THE INVENTION

The Brain Portal is a cylindrical device with a top cap, preferably made out of a plastic that is semi-rigid in nature that is slightly compressible. The device itself without attachments is approximately 0.25 inches long, and 0.25 inches in diameter, with a slightly tapered end. Note the diameter is the same size as the standard 0.25 inch drill bit. Compression is used to hold the device in place and to prevent the introduction of harmful pathogens. It can also be made with ribs and a slightly larger diameter, so the ribs take up the compression.

That is why the device must be semi-rigid in its material construction. A non semi-rigid material would be too hard and would not compress, and would possibly damage the skull. On one end is a small cap which allows for easy handling of the device. On the opposite end is a tapered receptacle for an optional extension which can be used to deliver medication deep within the brain. The inside of the cap is hollow, and contains one or more membranes that are self-sealing when punctured by a needle.

The optional extension is a hollow tube with a ball shaped structure on one end, and tapers to the final diameter of a brain probe. The interior of the extension is hollow, allowing a brain probe to be slipped into the device. The optional extension is made of the same semi rigid material as the Brain Portal described earlier. Installation of the extension to the Brain Portal involves snapping the two pieces together, with the ball part of the extension fitting into the hollow receptacle on the Brain Portal.

The Brain Portal can also be manufactured so as to be an electrical stimulator for the brain. The Membrane is substituted by a solid plug, made from the same semi-rigid material, and has metal electrodes attached along with an induction coil to carry the current.

### MANUFACTURE

The Brain Portal is a very simple, uncomplicated device that can be manufactured in a variety of ways. Primary consideration should be made to use injection molding processes. Injection molding can be used for both the Brain Portal and its extension, and with some slight modification, can also be used for the electrical stimulator.

### SPECIFICATIONS

FIG. 1 depicts a side cross-sectional view of the Brain Portal with its component parts. Part A in FIG. 1 depicts the top cap, with the dotted lines indicating a hole thru the structure. The top cap is approximately 0.35 inches wide and 0.1 inch in height, with a center hole of 0.15 inches in diameter.

Part B in FIG. 1 depicts the center hole, which runs down to the membrane (Part C, FIG. 1). The center hole continues after the membrane to the end of the device.

Part C in FIG. 1 depicts the membrane. More than one membrane can be used, if desired.

Part D in FIG. 1 depicts the spherical cavity that is used to link the Brain Portal to the extension piece shown in FIG. 2.

Part E depicts the side wall of the device. This side wall is approximately 0.05 inches thick. At the bottom of part E, the device tapers slightly to allow for easy insertion into the drilled hole in the skull.

FIG. 2 depicts the extension piece that is used to deliver medication deep into the brain.

Part A in FIG. 2 depicts the center hole thru the extension piece, which runs the length of the piece.

Part B in FIG. 2 depicts the ball that snaps into the end piece.

Part C in FIG. 2 depicts the flexible tube of varied length, which is commonly cut to the desired length.

FIG. 3 depicts the electrical stimulator embodiment of the Brain Portal.

Part A in FIG. 3 depicts the induction coil which is attached to the electrodes.

Part B in FIG. 3 depicts the top cap. The dimensions of the cap are essentially the same as those noted in FIG. 1. Note the top cap center hole has been filled with plastic in which the coil and the electrodes are embedded.

Part C in FIG. 3 depicts the side wall of the electrical stimulator device. The outside diameter of the device at this point is 0.25 inches, with a side all thickness of 0.05 inches. The center hole in FIG. 3 is empty to allow for compression as the device is inserted into the skull, allowing for a tight fit.

Part D in FIG. 3 depicts the electrodes that are attached to the induction coil, and continue past the device into the brain. These insulated electrodes are cut to the desired length by the surgeon.

### USE AND PROCEDURES FOR EMPLOYING THE BRAIN PORTAL

The Brain Portal was designed to be very simple to employ. After the patient is given a local anesthetic and the scalp cleaned with an antibiotic solution, a half-inch incision is made in the scalp over the part of the skull that the physician determines is optimal. In most instances, that part of the skull would be located on the top of the head. Retractors would be used to hold back the scalp, and then a medical drill would drill a 0.25 inch in diameter hole. Most skulls are approximately 0.25 inches thick.

The drill would be placed in a special apparatus to insure that the depth of the hole does not exceed the thickness of the skull at the drilling point.

Once the hole has been drilled, the drill is removed, any medical procedures accomplished, and the hole is plugged with the Brain Portal. Emplacement involves nothing more than pressing the Brain Portal with sufficient pressure to seat it in the hole. The semi-rigid nature of the device allows it to compress, ensuring a very tight fit in the hole. An antimicrobial lubricant can be used to facilitate placement, such as sterile honey.

Once the Brain Portal is in place, the scalp is sown together with a few stitches. The entire process should take approximately 30 minutes and can be done on an out-patient basis.

The extension of the Brain Portal is to be used by neurosurgeons, as it involves deep penetration of the brain to deliver medications to the affected parts. Employment is similar in nature to the process described above, with some variation. The extension is designed to be used with a brain probe. Prior diagnosis should have determined exactly where the device should be located, including how far the target area is to the bottom part of the scalp. This can easily be determined thru the analysis of x-rays or other devices. The extension is then cut by the physician to ensure the proper length. The probe is used to make a path to the area, and then the extension is carefully slipped over the probe and is placed in the area. The probe is then removed, leaving the extension in place. The easiest method to attach the Brain Portal to the extension is to snap the two together before threading both over the probe. The membrane(s) in the Brain Portal are self-sealing, so the probe will not harm them when it penetrates the membranes. Once the Brain Portal is in place, the scalp is closed and stitched.

The electrical embodiment of the Brain Portal is also emplaced by a neurosurgeon. With the electrical embodiment, the extension is not used, and the electrodes are placed in the correct area by the neurosurgeon. The electrodes have been pre-sized by the neurosurgeon before the operation, so the final emplacement involves pushing the electrical stimulator into the hole in the skull, and closing the wound. If the electrodes are the correct length or even a bit longer than anticipated, there is no problem, as the electrodes are flexible and will nest in the brain without causing harm. If the neurosurgeon finds the electrode length too short, he/she should replace the device with another with longer electrodes.

### BRIEF DESCRIPTION OF THE DRAWINGS

FIG. 1 illustrates an example of the cross-sectional yew of the Brain Portal, in accordance with certain embodiments of the present disclosure.

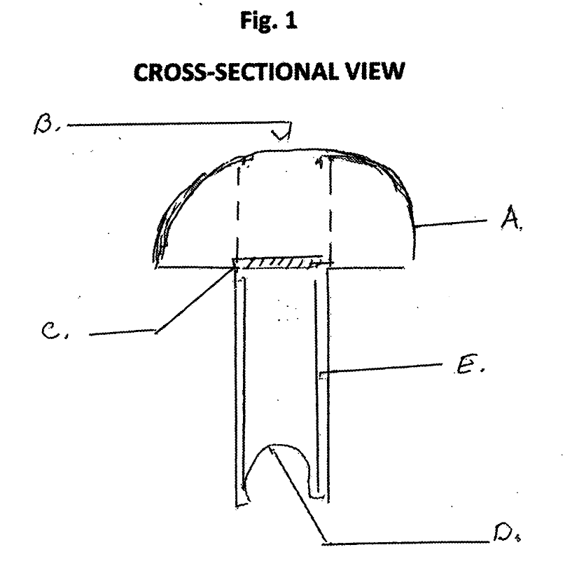

FIG. 2 illustrates an example of the extension piece, in accordance with certain embodiments of the present disclosure.

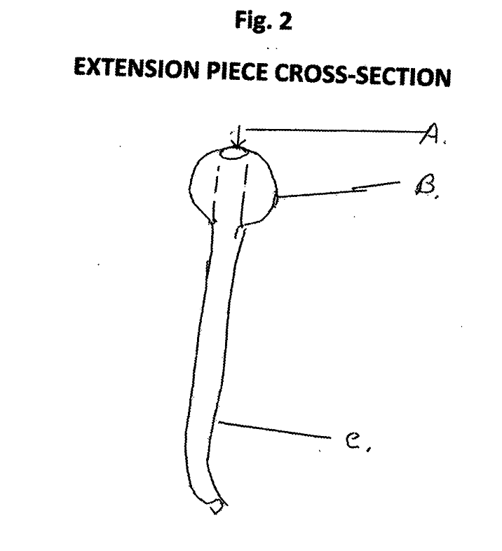

FIG. 3 illustrates an example of the electrical stimulator, in accordance with certain embodiments of the present disclosure.

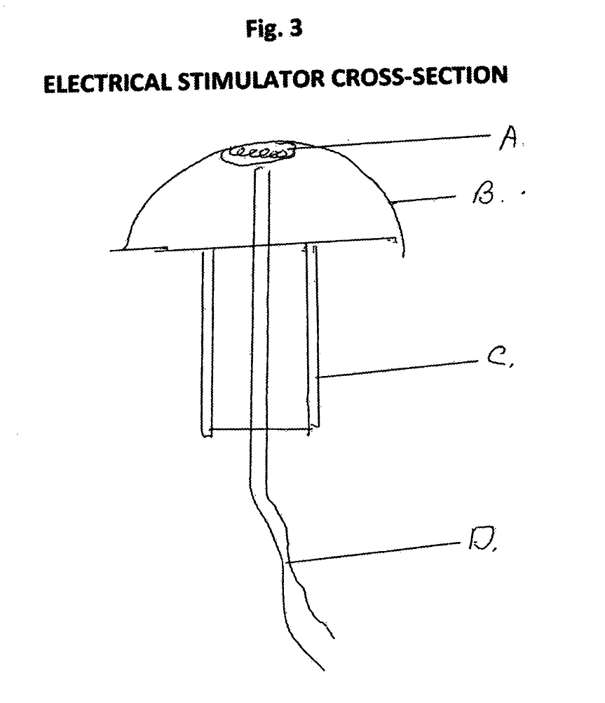

I claim:

1. A transcranial device preferably made from semi-rigid plastic with smooth sides or ridges and that allows multiple injections to be made thru the center of the device through one or more membranes that are self-sealing, thereby preventing the introduction into the brain of harmful pathogens.

2. An optional attachment to the device mentioned in claim 1 that enables medication to be delivered directly to the area of the brain that needs treatment and that attaches to the device described in claim 1 by snapping it onto the end closest to the brain.

3. An embodiment of the device mentioned in claim 1 that substitutes the membrane for a solid piece of plastic with electrodes and adds an induction coil to the top, allowing the transmission of electricity through the scalp into the desired area of the brain.
